SEQUENCE LISTING (90 sequences)

SEQ ID NO: 1 - protein, 10 aa, Rattus norvegicus
SFNSYELGSL

SEQ ID NO: 2 - protein, 11 aa, Rattus norvegicus
ALTTDRGKTLV

SEQ ID NO: 3 - protein, 14 aa, Rattus norvegicus
KAEFWLDLQPQAKV

SEQ ID NO: 4 - protein, 58 aa, Homo sapiens
PFRPKVKSPRPYSNFDQEFLNEKARLSYSDKNLIDSMDQSAFAGFSFVNPKFEHLLED

SEQ ID NO: 5 - protein, 10 aa, Rattus norvegicus
TFNSYELGSL

SEQ ID NO: 6 - protein, 10 aa, Rattus norvegicus
AFNSYELGSL

SEQ ID NO: 7 - protein, 10 aa, Rattus norvegicus
SFNSYELGTL

SEQ ID NO: 8 - protein, 10 aa, Rattus norvegicus
TFNSYELGTL

SEQ ID NO: 9 - protein, 10 aa, Rattus norvegicus
SYNSYELGSL

SEQ ID NO: 10 - protein, 10 aa, Rattus norvegicus
SFNSFELGSL

SEQ ID NO: 11 - protein, 9 aa, Rattus norvegicus
SNSYDLGSL

SEQ ID NO: 12 - protein, 10 aa, Rattus norvegicus
SFNSYELPSL

SEQ ID NO: 13 - protein, 10 aa, Rattus norvegicus
SFNSYEIGSY

SEQ ID NO: 14 - protein, 10 aa, Rattus norvegicus
SFNSYEVGSI

SEQ ID NO: 15 - protein, 10 aa, Rattus norvegicus
SFNSYELGSV

SEQ ID NO: 16 - protein, 10 aa, Rattus norvegicus
SFNSYELGSI

SEQ ID NO: 17 - protein, 10 aa, Rattus norvegicus
SFNSYEIGSL

SEQ ID NO: 18 - protein, 10 aa, Rattus norvegicus
SFNSYEVGSL

SEQ ID NO: 19 - protein, 10 aa, Rattus norvegicus
AFNSYEIGSL

SEQ ID NO: 20 - protein, 6 aa, Rattus norvegicus
YELGSL

SEQ ID NO: 21 - protein, 6 aa, Rattus norvegicus
YDLGSL

SEQ ID NO: 22 - protein, 6 aa, Rattus norvegicus
FDLGSL

SEQ ID NO: 23 - protein, 6 aa, Rattus norvegicus
YDIGSL

SEQ ID NO: 24 - protein, 4 aa, Rattus norvegicus
IGSL

SEQ ID NO: 25 - protein, 6 aa, Rattus norvegicus
YDYGSL

SEQ ID NO: 26 - protein, 6 aa, Rattus norvegicus
YDLPSL

SEQ ID NO: 27 - protein, 6 aa, Rattus norvegicus
YDLGIL

SEQ ID NO: 28 - protein, 6 aa, Rattus norvegicus
YDLGSI

SEQ ID NO: 29 - protein, 6 aa, Rattus norvegicus
YDLGSV

SEQ ID NO: 30 - protein, 4 aa, Rattus norvegicus
LGSL

SEQ ID NO: 31 - protein, 4 aa, Rattus norvegicus
IGSL

SEQ ID NO: 32 - protein, 4 aa, Rattus norvegicus
VGSL

SEQ ID NO: 33 - protein, 4 aa, Rattus norvegicus
LPSL

SEQ ID NO: 34 - protein, 4 aa, Rattus norvegicus
LGIL

SEQ ID NO: 35 - protein, 4 aa, Rattus norvegicus
LGSI

SEQ ID NO: 36 - protein, 4 aa, Rattus norvegicus
LGSV

SEQ ID NO: 37 - protein, 11 aa, Rattus norvegicus
ALSTDRGKTLV

SEQ ID NO: 38 - protein, 11 aa, Rattus norvegicus
ALTSDRGKTLV

SEQ ID NO: 39 - protein, 11 aa, Rattus norvegicus
ALTTDRGKSLV

SEQ ID NO: 40 - protein, 11 aa, Rattus norvegicus
ALTTDRPKTLV

SEQ ID NO: 41 - protein, 11 aa, Rattus norvegicus
ALTTDRGRTLV

SEQ ID NO: 42 - protein, 11 aa, Rattus norvegicus
ALTTDKGKTLV

SEQ ID NO: 43 - protein, 11 aa, Rattus norvegicus
ALTTDKGKTLV

SEQ ID NO: 44 - protein, 14 aa, Rattus norvegicus
RAEFWLDLQPQAKV

SEQ ID NO: 45 - protein, 14 aa, Rattus norvegicus
KADFWLDLQPQAKV

SEQ ID NO: 46 - protein, 14 aa, Rattus norvegicus
KAEFWLELQPQAKV

SEQ ID NO: 47 - protein, 14 aa, Rattus norvegicus
KAEFWLDLQPQARV

SEQ ID NO: 48 - protein, 14 aa, Rattus norvegicus
KAEYWLDLQPQAKV

SEQ ID NO: 49 - protein, 14 aa, Rattus norvegicus
KAEFWIDLQPQAKV

SEQ ID NO: 50 - protein, 14 aa, Rattus norvegicus
KAEFWVDLQPQAKV

SEQ ID NO: 51 - protein, 14 aa, Rattus norvegicus
KAEFWLDIQPQAKV

SEQ ID NO: 52 - protein, 14 aa, Rattus norvegicus
KAEFWLDVQPQAKV

SEQ ID NO: 53 - protein, 14 aa, Rattus norvegicus
KAEFWLDLNPQAKV

SEQ ID NO: 54 - protein, 14 aa, Rattus norvegicus
KAEFWLDLQPNAKV

SEQ ID NO: 55 - protein, 14 aa, Rattus norvegicus
KAEFWLDLQPQAKI

SEQ ID NO: 56 - protein, 14 aa, Rattus norvegicus
KAEFWLDLQPQAKL

SEQ ID NO: 57 - protein, 14 aa, Rattus norvegicus
KAEFWADLQPQAKV

SEQ ID NO: 58 - protein, 14 aa, Rattus norvegicus
KAEFWLDAQPQAKV

SEQ ID NO: 59 - protein, 14 aa, Rattus norvegicus
KAEFWLDLQPQAKA

SEQ ID NO: 60 - protein, 7 aa, Rattus norvegicus
KAEFWLD

SEQ ID NO: 61 - protein, 8 aa, Rattus norvegicus
DLQPQAKV

SEQ ID NO: 62 - protein, 8 aa, Rattus norvegicus
EFWLDLQP

SEQ ID NO: 63 - protein, 7 aa, Rattus norvegicus
LDLQPQA

SEQ ID NO: 64 - protein, 7 aa, Rattus norvegicus
LQPQAKV

SEQ ID NO: 65 - protein, 7 aa, Rattus norvegicus
AEFWLDL

SEQ ID NO: 66 - protein, 7 aa, Rattus norvegicus
WLDLQPQ

SEQ ID NO: 67 - protein, 8 aa, Homo sapiens
SPRPYSNF

SEQ ID NO: 68 - protein, 8 aa, Homo sapiens
RPYSNFDQ

SEQ ID NO: 69 - protein, 8 aa, Homo sapiens
SNFDQEFL

SEQ ID NO: 70 - protein, 8 aa, Homo sapiens
DQEFLNEK

SEQ ID NO: 71 - protein, 8 aa, Homo sapiens
FLNEKARL

SEQ ID NO: 72 - protein, 8 aa, Homo sapiens
LIDSMDQS

SEQ ID NO: 73 - protein, 8 aa, Homo sapiens
SMDQSAFA

SEQ ID NO: 74 - protein, 8 aa, Homo sapiens
DQSAFAGF

SEQ ID NO: 75 - protein, 8 aa, Homo sapiens
FVNPKFEH

SEQ ID NO: 76 - protein, 8 aa, Homo sapiens
KFEHLLED

SEQ ID NO: 77 - protein, 8 aa, Homo sapiens
NEKARLSY

SEQ ID NO: 78 - protein, 8 aa, Homo sapiens
RLSYSDKN

SEQ ID NO: 79 - protein, 8 aa, Homo sapiens
SYSDKNLI

SEQ ID NO: 80 - protein, 8 aa, Homo sapiens
DKNLIDSM

SEQ ID NO: 81 - protein, 8 aa, Homo sapiens
PFRPKVKS

SEQ ID NO: 82 - protein, 8 aa, Homo sapiens
RPKVKSPR

SEQ ID NO: 83 - protein, 8 aa, Homo sapiens
VKSPRPYS

SEQ ID NO: 84 - protein, 17 aa, Drosophila Antennapedia homeodomain
CRQIKIWFQNRRMKWKK

SEQ ID NO: 85 - protein, 11 aa, Human immunodeficiency virus 1
YGRKKRRQRRR

SEQ ID NO: 86 - protein, 23 aa, Artificial Sequence
  Synthetic Construct
YGRKKRRQRRRCCSFNSYELGSL

SEQ ID NO: 87 - protein, 8 aa, Rattus norvegicus
EAVSLKPT

SEQ ID NO: 88 - protein, 21 aa, Artificial Sequence
  Synthetic Construct
YGRKKRRQRRRCCEAVSLKPT

SEQ ID NO: 89 - protein, 52 aa, Rattus norvegicus
PKARDKRDTSNFDKEFTRQPVELTPTDKLFIMNLDQNEFAGFSYTNPEFVIN

SEQ ID NO: 90 - protein, 55 aa, Rattus norvegicus
PKACGRNAENFDRFFTRHPPVLTPPDQEVIRNIDQSEFEGFSFVNSEFLKPEVKS

(19) United States
(12) Patent Application Publication
(10) Pub. No.: US 2007/0066526 A1
(43) Pub. Date: Mar. 22, 2007

(54) Method for reducing risk of and extent of injury due to stroke in hypertensive subjects

(76) Inventors: Daria Mochly-Rosen, Menlo Park, CA (US); Koichi Inagaki, Otsu (JP); Rachel Bright, Claremont, CA (US)

Correspondence Address: PERKINS COIE LLP, P.O. BOX 2168, MENLO PARK, CA, 94026, US

(21) Appl. No.: 11/514,666

(22) Filed: Sep. 1, 2006

Related U.S. Application Data
(60) Provisional application No. 60/713,699, filed on Sep. 2, 2005.

(51) Int. Cl. A61K 38/55

(52) U.S. Cl. 514/12

(57) ABSTRACT

A method for reducing the risk of stroke in a subject with hypertension is described. The method includes administering an inhibitor of delta protein kinase C (PKC) to the subject. Also described is a method for improving the survival from stroke in a subject with chronic hypertension by treating the subject with an inhibitor of delta PKC.

### CROSS-REFERENCE TO RELATED APPLICATIONS

This application claims the benefit of U.S. Provisional Application No. 60/713,699, filed Sep. 2, 2005, incorporated herein by reference in its entirety.

### STATEMENT REGARDING GOVERNMENT INTEREST

This work was supported in part by the National Institutes of Health under grant numbers HL076675 and NS044350. Accordingly the United States government may have certain rights in this invention.

### TECHNICAL FIELD

The subject matter described herein relates to a method of reducing the risk of stroke in persons with hypertension and to a method of improving survival from stroke in such persons.

### BACKGROUND

Stroke involves damage to part of the brain caused by interruption to its blood supply or leakage of blood outside of vessel walls. Sensation, movement, or function controlled by the damage area is impaired after a stroke. Stoke is a source of serious disability and mortality and the overall death rate for stroke is approximately 58%, with about 50% of these patients dying in a hospital. In addition, over one million people in the U.S. currently live with functional disabilities following stroke (American Heart Association Heart Disease and Stroke Statistics, Updated 2004).

Stroke may be caused by cerebral thrombosis, cerebral embolism, or hemorrhage. Cerebral thrombosis results from blockage by a thrombus (clot) that has built up on the wall of a brain artery. A cerebral embolism involves blockage by an embolus (usually a clot) swept into an artery in the brain. Rupture of a blood vessel in or near the brain may cause an intracerebral hemorrhage or a subarachnoid hemorrhage.

Since any part of the brain may be affected by a stroke, the symptoms of stroke vary accordingly. Generally, symptoms of a stroke develop over minutes or hours, but occasionally over several days. Depending on the site, cause, and extent of damage, any or all of the symptoms of headache, dizziness and confusion, visual disturbance, slurred speech or loss of speech, and/or difficult swallowing, may be present. In more serious cases, a rapid loss of consciousness, coma, and death can occur, or severe physical or mental handicap may result.

Certain factors increase the risk of stroke. One of the more important risk factors is hypertension, which weakens the walls of arteries. Hypertension, or high blood pressure, refers to the pressure of blood in the main arteries of the body. Blood pressure goes up as a normal response to stress and physical activity, however, a person with hypertension has a high blood pressure at rest. A high blood pressure at rest is typically defined as a resting blood pressure greater than 140 mm Hg (systolic)/90 mm Hg (diastolic). Persons suffering from chronic arterial hypertension typically have an unimpaired oxygen consumption and cerebral blood flow in the resting state. However, alterations in vascular autoregulation contribute to reduced tolerance to changes in arterial blood pressure, increased susceptibility to vasoconstrictive agents produced following cerebral ischemia, and impaired endothelium-dependent dilation (Strandgaard, S., Acta Neurol Scand Suppl., 66:1-82 (1978); Chillon J-M. et al., Autoregulation: Arterial and Intracranial Pressure, 2^{nd} Ed. Philadelphia: Lippincoft Williams & Wilkins, 2002). Thus, hypertension results in increased susceptibility to ischemic and hemorrhagic stroke, and worsened outcome following such an event (Rasool, A. H. et al. J. Hum. Hypertens., 18:187-92 (2004); Arboix, A. et al., Eur. J. Neurol., 11:687-92 (2004)). Although multiple anti-hypertensive drugs exist, agents that protect cerebral microvascular function and confer neuroprotection following an ischemic event have not been identified.

Protein kinase C (PKC) has been shown to regulate cerebrovascular tone. (Uhl, M. W. et al., Stroke., 24:1977-82 (1993); Murray, M. A. et al., J. Physiol., 445:169-79 (1992); Laher, I. et al., J. Cereb. Blood Flow Metab., 21:887-906 (2001)) PKC activator phorbol esters have been shown to induce vasoconstriction in multiple ex vivo and in vivo systems, (Uhl, M. W. et al., Stroke., 24:1977-82 (1993); Murray, M. A. et al., J. Physiol., 445:169-79 (1992); Akopov, S. E. et al., J. Cereb. Blood Flow Metab., 14:1078-87 (1994); Jin, Y. et al., J. Neurosurg., 81:574-8 (1994)) and pan-specific PKC inhibitors, including staurosporine, calphostin C, and H-7 promote vasodilation or block vasoconstriction in isolated arteries. (Murray, M. A. et al., J. Physiol., 445:169-79 (1992); Henrion, D. et al., Can J Physiol Pharmacol., 71:521-4 (1993)). The exact mechanism by which PKC elicits increased blood flow is unclear, in part, due to a lack of understanding about the role of individual PKC isozymes.

PKC isozymes mediate unique cellular functions in response to cellular stresses such as ischemia. Delta PKC in particular mediates cellular damage following ischemic/reperfusion damage in multiple organs. (La Porta, C. A. et al., Biochem Biophys Res Commun., 191:1124-30 (1993); Bright, R. et al., J. Neurosci., 24:6880-88 (2004)). Inhibition or reduction of delta PKC isozyme levels during reperfusion leads to marked reduction in cerebral damage. (Bright, R. et al., J. Neurosci., 24:6880-88 (2004); Raval, A. P., et al., J. Cereb. Blood Flow Metab., 25:730-41 (2005)). This protection is due to inhibition of deleterious delta PKC activity in multiple cell types including parenchymal and inflammatory cells following stroke (Bright, R. et al., J. Neurosci., 24:6880-88 (2004); Chou, W. H., et al., J Clin Invest., 114:49-56 (2004)). In addition, the levels of delta PKC increase in endothelial cells 2-3 days following ischemic/reperfusion injury in vivo (Miettinen, S. et al., J Neurosci., 16:6236-45 (1996)). Whether delta PKC specifically mediates acute cerebrovascular responses during reperfusion injury, thereby contributing to stroke damage, is unknown.

The foregoing examples of the related art and limitations related therewith are intended to be illustrative and not exclusive. Other limitations of the related art will become apparent to those of skill in the art upon a reading of the specification and a study of the drawings.

### SUMMARY

The following aspects and embodiments thereof described and illustrated below are meant to be exemplary and illustrative, not limiting in scope.

In one aspect, a method for reducing the risk of stroke in a subject with hypertension is provided, the method comprising administering an inhibitor of delta protein kinase C (δPKC) to the subject. The subject can be suffering from chronic hypertension or from acute hypertension.

In one embodiment, the δPKC inhibitor is a peptide. An exemplary peptide is one having at least about 50% identity to the amino acid sequence of delta V1-1 set forth in SEQ ID NO:1. In another embodiment, the δPKC inhibitor is a peptide having an amino acid sequence as set forth in SEQ ID NO:1.

In embodiments where the inhibitor is a peptide, the peptide can be linked to a moiety effective to facilitate transport across a cell membrane. Such linking can be facilitated, if desired, by modifying the peptide to include at least one terminal cysteine residue; either a C-terminal or N-terminal residue. For example, and in one embodiment, a δPKC inhibitor peptide can be Cys-Cys bonded to a carrier peptide selected from poly-Arg, Tat, or the Drosophila Antennapedia homeodomain.

In yet another embodiment, the method includes administering a pharmaceutical formulation comprising a pharmaceutically-acceptable excipient and a peptide inhibitor of δPKC. Administration can be via any suitable route known in the medical field, and includes, but is not limited to parenteral, subcutaneous, inhalation, nasal, etc. Subcutaneous administration may be achieved via an implanted osmotic pump.

Administration of the δPKC inhibitor can be on a chronic or sustained basis.

In another aspect, a method for improving the survival from stroke in a subject with chronic hypertension is disclosed. The method comprises administering an inhibitor of δPKC to the subject.

In addition to the exemplary aspects and embodiments described above, further aspects and embodiments will become apparent by reference to the drawings and by study of the following descriptions.

### BRIEF DESCRIPTION OF THE DRAWINGS

FIG. 1A is a plot of cerebral blood flow, as a percent of baseline flow, over a time course in animals treated with δV1-1-Tat peptide intraperitoneally;

FIG. 1B is a scattergram plot showing the average change in cerebral blood flow from baseline flow for animals treated with an intraperitoneal injection of Tat alone or with δV1-1-Tat;

FIG. 2A is a Western blot of δPKC levels in brain tissue from animals treated with Tat alone or with δV1-1-Tat; the brain tissue was fractionated into soluble (cystolic) and particulate (membrane) fractions and probed with an anti-6PKC antibody;

FIG. 2B is a plot of δPKC activation in rat brain, expressed as percent of δPKC translocation from the soluble to membrane-bound fraction, and determined by the density of the bands in FIG. 2A, in rats treated with Tat alone or with δV1-1-Tat;

FIG. 3A is a Western blot of εPKC levels in brain tissue from animals treated with Tat alone or with δV1-1-Tat, the brain tissue fractionated into soluble (cystolic) and particulate (membrane) fractions and probed with an anti-εPKC antibody;

FIG. 3B is a plot of εPKC activation, expressed as a percent of translocation, and determined by the density of the bands in FIG. 3A, from the brain tissue of rats treated with Tat alone or with δV1-1-Tat;

FIG. 4A is a representative photomicrograph of triphenyl-tetrazolium chloride (TTC) treated brain slice with cerebral infarction;

FIG. 4B is a graph showing the average infarct size, measured from photomicrographs of brain slices of each animal, in animals treated with Tat alone or with δV1-1-Tat; and

FIG. 5 is a graph showing the percentage of animals surviving as a function of animal age, in weeks, for animals treated beginning at 11 weeks of age with saline, Tat, δV1-1-Tat, or εV1-2-Tat.

### BRIEF DESCRIPTION OF THE SEQUENCES

SEQ ID NO:1 is an amino acid sequence corresponding to amino acid residues 8-17 of Rattus norvegicus δPKC, as found in Genbank Accession No. MH76505, and referred to herein as δV1-1.

SEQ ID NO: 2 is an amino acid sequence corresponding to amino acid residsues 35 to 45 of Rattus norvegicus δPKC, as found in Genbank Accession No. MH76505, and referred to herein as δV1-2.

SEQ ID NO: 3 is an amino acid sequence corresponding to amino acid residues 569 to 626 of Rattus norvegicus δPKC, as found in Genbank Accession No. MH76505, and referred to herein as δV1-5.

SEQ ID NO: 4 is an amino acid sequence corresponding to amino acid residues 561-626 of human δPKC, as found in Genbank Accession No. BM01381, with the exception that amino acid 11 (aspartic acid) is substituted with a proline.

SEQ ID NO:5 is a modification of δV1-1 (SEQ ID NO:1).

SEQ ID NO:6 is a modification of δV1-1 (SEQ ID NO:1).

SEQ ID NO:7 is a modification of δV1-1 (SEQ ID NO:1).

SEQ ID NO:8 is a modification of δV1-1 (SEQ ID NO:1).

SEQ ID NO:9 is a modification of δV1-1 (SEQ ID NO:1).

SEQ ID NO:10 is a modification of δV1-1 (SEQ ID NO:1).

SEQ ID NO:11 is a modification of δV1-1 (SEQ ID NO:1).

SEQ ID NO:12 is a modification of δV1-1 (SEQ ID NO:1).

SEQ ID NO:13 is a modification of δV1-1 (SEQ ID NO:1).

SEQ ID NO:14 is a modification of δV1-1 (SEQ ID NO:1).

SEQ ID NO:15 is a modification of δV1-1 (SEQ ID NO:1).

SEQ ID NO:16 is a modification of δV1-1 (SEQ ID NO:1).

SEQ ID NO:17 is a modification of δV1-1 (SEQ ID NO:1).

SEQ ID NO:18 is a modification of δV1-1 (SEQ ID NO:1).

SEQ ID NO:19 is a modification of δV1-1 (SEQ ID NO:1).

SEQ ID NO:20 is a fragment of δV1-1 (SEQ ID NO:1).

SEQ ID NO:21 is a fragment of δV1-1 (SEQ ID NO:1).

SEQ ID NO:22 is a fragment of δV1-1 (SEQ ID NO:1).

SEQ ID NO:23 is a fragment of δV1-1 (SEQ ID NO:1).

SEQ ID NO:24 is a fragment of δV1-1 (SEQ ID NO:1).

SEQ ID NO:25 is a fragment of δV1-1 (SEQ ID NO:1).

SEQ ID NO:26 is a fragment of δV1-1 (SEQ ID NO:1).

SEQ ID NO:27 is a fragment of δV1-1 (SEQ ID NO:1).

SEQ ID NO:28 is a fragment of δV1-1 (SEQ ID NO:1).

SEQ ID NO:29 is a fragment of δV1-1 (SEQ ID NO:1).

SEQ ID NO:30 is a fragment of δV1-1 (SEQ ID NO:1).

SEQ ID NO:31 is a fragment of δV1-1 (SEQ ID NO:1).

SEQ ID NO:32 is a fragment of δV1-1 (SEQ ID NO:1).

SEQ ID NO:33 is a fragment of δV1-1 (SEQ ID NO:1).

SEQ ID NO:34 is a fragment of δV1-1 (SEQ ID NO:1).

SEQ ID NO:35 is a fragment of δV1-1 (SEQ ID NO:1).

SEQ ID NO:36 is a fragment of δV1-1 (SEQ ID NO:1).

SEQ ID NO:37 is a modification of δV1-2 (SEQ ID NO:2).

SEQ ID NO:38 is a modification of δV1-2 (SEQ ID NO:2).

SEQ ID NO:39 is a modification of δV1-2 (SEQ ID NO:2).

SEQ ID NO:40 is a modification of δV1-2 (SEQ ID NO:2).

SEQ ID NO:41 is a modification of δV1-2 (SEQ ID NO:2).

SEQ ID NO:42 is a modification of δV1-2 (SEQ ID NO:2).

SEQ ID NO:43 is a modification of δV1-2 (SEQ ID NO:2).

SEQ ID NO:44 is a modification of δV1-5 (SEQ ID NO:3).

SEQ ID NO:45 is a modification of δV1-5 (SEQ ID NO:3).

SEQ ID NO:46 is a modification of δV1-5 (SEQ ID NO:3).

SEQ ID NO;47 is a modification of δV1-5 (SEQ ID NO:3).

SEQ ID NO:48 is a modification of δV1-5 (SEQ ID NO:3).

SEQ ID NO:49 is a modification of δV1-5 (SEQ ID NO:3).

SEQ ID NO:50 is a modification of δV1-5 (SEQ ID NO:3).

SEQ ID NO:51 is a modification of δV1-5 (SEQ ID NO:3).

SEQ ID NO:52 is a modification of δV1-5 (SEQ ID NO:3).

SEQ ID NO:53 is a modification of δV1-5 (SEQ ID NO:3).

SEQ ID NO:54 is a modification of δV1-5 (SEQ ID NO:3).

SEQ ID NO:55 is a modification of δV1-5 (SEQ ID NO:3).

SEQ ID NO:56 is a modification of δV1-5 (SEQ ID NO:3).

SEQ ID NO:57 is a modification of δV1-5 (SEQ ID NO:3).

SEQ ID NO:58 is a modification of δV1-5 (SEQ ID NO:3).

SEQ ID NO:59 is a modification of δV1-5 (SEQ ID NO:3).

SEQ ID NO:60 is a fragment of δV1-5 (SEQ ID NO:3).

SEQ ID NO:61 is a fragment of δV1-5 (SEQ ID NO:3).

SEQ ID NO:62 is a fragment of δV1-5 (SEQ ID NO:3).

SEQ ID NO:63 is a fragment of δV1-5 (SEQ ID NO:3).

SEQ ID NO:64 is a fragment of δV1-5 (SEQ ID NO:3).

SEQ ID NO:65 is a fragment of δV1-5 (SEQ ID NO:3).

SEQ ID NO:66 is a fragment of δV1-5 (SEQ ID NO:3).

SEQ ID NO:67 is a fragment of δV5 (SEQ ID NO:4).

SEQ ID NO:68 is a fragment of δV5 (SEQ ID NO:4).

SEQ ID NO:69 is a fragment of δV5 (SEQ ID NO:4).

SEQ ID NO:70 is a fragment of δV5 (SEQ ID NO:4).

SEQ ID NO:71 is a fragment of δV5 (SEQ ID NO:4).

SEQ ID NO:72 is a fragment of δV5 (SEQ ID NO:4).

SEQ ID NO:73 is a fragment of δV5 (SEQ ID NO:4).

SEQ ID NO:74 is a fragment of δV5 (SEQ ID NO:4).

SEQ ID NO:75 is a fragment of δV5 (SEQ ID NO:4).

SEQ ID NO:76 is a fragment of δV5 (SEQ ID NO:4).

SEQ ID NO:77 is a fragment of δV5 (SEQ ID NO:4).

SEQ ID NO:78 is a fragment of δV5 (SEQ ID NO:4).

SEQ ID NO:79 is a fragment of δV5 (SEQ ID NO:4).

SEQ ID NO:80 is a fragment of δV5 (SEQ ID NO:4).

SEQ ID NO:81 is a fragment of δV5 (SEQ ID NO:4).

SEQ ID NO:82 is a fragment of δV5 (SEQ ID NO:4).

SEQ ID NO:83 is a fragment of δV5 (SEQ ID NO:4).

SEQ ID NO:84 is the amino acid sequence from Drosophila Antennapedia homeodomain.

SEQ ID NO:85 is an amino acid sequence from the Transactivating Regulatory Protein (Tat; Genbank Accession No. MT48070), corresponding to residues 47-57 of Tat.

SEQ ID NO:86 is a conjugate of δV1 -1 (SEQ ID NO:1) joined to a TAT carrier peptide (SEQ ID NO:85) through an N-terminal cysteine-cysteine bond, also referred to herein as δV1-1-Tat.

SEQ ID NO:87 is an eight amino acid peptide derived from εPKC, and is referred to herein as εV1-2.

SEQ ID NO:88 is a conjugate of SEQ ID NO:87 joined to a TAT carrier peptide (SEQ ID NO:85) through an N-terminal cysteine-cysteine bond, also referred to herein as εV1-2-Tat.

SEQ ID NO:89 is the amino acid sequence of the V5 domain of the βI-PKC isozyme.

SEQ ID NO:90 is the amino acid sequence of the V5 domain of the βII-PKC isozyme.

### DETAILED DESCRIPTION

### I. Definitions

It must be noted that as used herein and in the appended claims, the singular forms “a”, “an”, and “the” include plural reference unless the context clearly dictates otherwise.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All publications mentioned herein are incorporated herein by reference for the purpose of describing and disclosing the methodologies which are reported in the publications which might be used in connection with the invention.

Protein sequences are presented herein using the one letter or three letter amino acid symbols as commonly used in the art and in accordance with the recommendations of the IUPAC-IUB Biochemical Nomenclature Commission.

The term “substantially purified”, as used herein, refers to nucleic or amino acid sequences that are removed from their natural environment, isolated or separated, and are at least 60% free, preferably 75% free, more preferably 90% free, and most preferably 95% free from other components with which they are naturally associated.

“Peptide” and “polypeptide” are used interchangeably herein and refer to a compound made up of a chain of amino acid residues linked by peptide bonds. Unless otherwise indicated, the sequence for peptides is given in the order from the amino terminus to the carboxyl terminus.

A “substitution”, as used herein, refers to the replacement of one or more amino acids by different amino acids, respectively.

An “insertion” or “addition”, as used herein, refers to a change in an amino acid sequence resulting in the addition of one or more amino acid residues, as compared to the naturally occurring molecule.

A “deletion”, as used herein, refers to a change in the amino acid sequence and results in the absence of one or more amino acid residues.

A “variant” of a first amino acid sequence refers to a second amino acid sequence that has one or more amino acid substitutions or deletions, relative to the first amino acid sequence.

A “modification” of an amino acid sequence or a “modified” amino acid sequence refers to an amino acid sequence that results from the addition of one or more amino acid residues, to either the N-terminus or the C-terminus of the sequence.

The term “modulate”, as used herein, refers to a change in the activity of delta protein kinase C. For example, modulation may cause an increase or a decrease in protein activity, binding characteristics, or any other biological, functional or immunological properties of delta PKC.

Reference herein to an “amino acid sequence having ‘x’ percent identity” with another sequence intends that the sequences have the specified percent identity, ‘x’, determined as set forth below, and share a common functional activity. To determine the percent identity of two amino acid sequences, the sequences are aligned for optimal comparison purposes (e.g., gaps can be introduced in one or both of a first and a second amino acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 75%, 80%, 85%, 90%, or 95% of the length of the reference sequence. For the relatively short peptide sequences described herein, percent identity is taken as the number of like residues between the first and second sequence relative to the total number of residues in the longer of the first and second sequences. The comparison of sequences and determination of percent identity between two sequences can also be accomplished using a mathematical algorithm. The percent identity between two amino acid sequences can be determined using the Needleman and Wunsch (J. Mol. Biol., 48:444-453 (1970)) algorithm which has been incorporated into the GAP program in the GCG software package (available at http://www.gcg.com), using either a Blosum 62 matrix or a PAM250 matrix, and a gap weight of 16, 14, 12, 10, 8, 6, or 4 and a length weight of 1, 2, 3, 4, 5, or 6. The percent identity between two amino acid sequences can also be determined using the algorithm of E. Meyers and W. Miller (CABIOS, 4:11-17 (1989)) which has been incorporated into the ALIGN program (version 2.0), using a PAM120 weight residue table, a gap length penalty of 12 and a gap penalty of 4. Protein sequences can further be used as a “query sequence” to perform a search against public databases; for example, BLAST protein searches can be performed with the XBLAST program, score=50, wordlength=3. See http://www.ncbi.nlm.nih.gov.

“Ischemia” is defined as an insufficient supply of blood to a specific organ or tissue. A consequence of decreased blood supply is an inadequate supply of oxygen to the organ or tissue (hypoxia). Prolonged hypoxia may result in injury to the affected organ or tissue.

“Anoxia” refers to a virtually complete absence of oxygen in the organ or tissue, which, if prolonged, may result in death of the organ or tissue.

“Hypoxic condition” is defined as a condition under which a particular organ or tissue receives an inadequate supply of oxygen.

“Anoxic condition” refers to a condition under which the supply of oxygen to a particular organ or tissue is cut off.

“Ischemic injury” refers to cellular and/or molecular damage to an organ or tissue as a result of a period of ischemia.

“Hypertension” or “high blood pressure” refers to a resting blood pressure, as measured with a sphygmomanometer, of greater than 120 mmHg (systolic)/80 mmHg (diastolic). Blood pressure between 121-139/81-89 is considered prehypertension and above this level (140/90 mm Hg or higher) is considered high (hypertension). Both prehypertension and hypertension blood pressure are included in the meaning of “hypertension” as used herein. For example, resting blood pressures of 135 mmHg/87 or of 140 mmHg/90 mmHg are intended to be within the scope of the term “hypertension” even though the 135/87 is within a prehypertensive category. Blood pressures of 145 mm Hg/90 mmHg, 140 mmHg/95 mmHg, and 142 mmHg/93 mmHg are further examples of high blood pressures. It will be appreciated that blood pressure normally varies throughout the day. It can even vary slightly with each heartbeat. Normally, it increases during activity and decreases at rest. It's often higher in cold weather and can rise when under stress. More accurate blood pressure readings can be obtained by daily monitoring blood pressure, where the blood pressure reading is taken at the same time each day to minimize the effect that external factors. Several readings over time may be needed to determine whether blood pressure is high.

### II. Method of Treatment

Accordingly, a method for reducing the risk of stroke in hypertensive subjects is provided by administering an agonist (inhibitor) of delta PKC (δPKC). Also described is a method for improving the survival from stroke in subjects with hypertension by treating the subject with an inhibitor of δPKC. Patients suitable for treatment can have either chronic or acute hypertension.

A variety of compounds can act as inhibitors of δPKC, and specific examples are given below. By inhibitor of δPKC, it is meant herein a compound that inhibits the biological activity or function of δPKC. As known in the art, δPKC is involved a myriad of cellular processes, including regulation of cell growth, and regulation of gene expression. The inhibitors may, for example, inhibit the enzymatic activity of δPKC. The inhibitors may inhibit the activity of δPKC by, for example, preventing activation of δPKC or preventing binding of δPKC to its protein substrate. Such an inhibition of enzymatic activity would prevent, for example, phosphorylation of amino acids in proteins. The inhibitor may also prevent binding of δPKC to its receptor for activated kinase (RACK), or any other anchoring protein, and subsequent translocation of δPKC to its subcellular location.

The methods described herein are illustrated using a peptide inhibitor of δPKC. However, it will be appreciated that the peptide inhibitor is merely exemplary, and that other compounds, peptides and non-peptides, may be similarly suitable. In the particular studies now to be described, a δPKC peptide referred to as δV1-1 and identified herein as SEQ ID NO:1 was used to illustrate the beneficial effects of δPKC inhibition in hypertensive subjects.

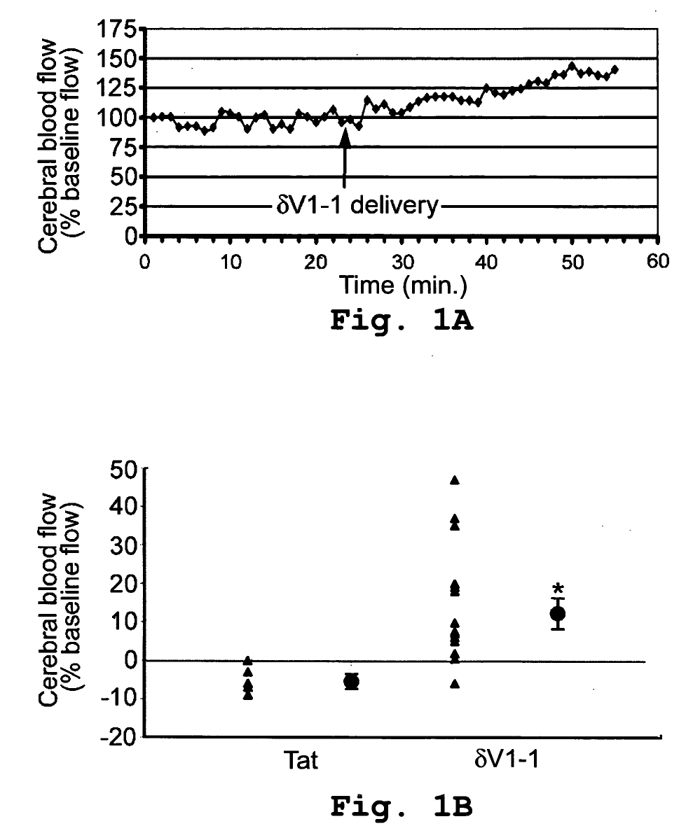

In a first study, a Dahl salt-sensitive rat model was used to illustrate that delivery of δV1-1 increases cerebral blood flow in hypertensive subjects. The Dahl salt-sensitive rat model is a well established model of hypertension (Iwai, J. et al., J. Hypertens. Suppl., 4:S29-31 (1986). Fed an 8% high-salt diet starting at 6 weeks of age, these rats rapidly develop hypertension, and by 11 weeks their blood pressure stabilizes at 233±4 mmHg, as compared to 156±4 mmHg in animals fed on a 0.3% low-salt diet (Inagaki, K. et al., J. Mol. Cell Cardiol, 34:1377-85 (2002)). As described in Example 1, the effects of δV1-1-Tat treatment on cerebral blood flow in Dahl hypertensive rats at age 11 weeks, following 5 weeks of high-salt diet, was assessed. The animals were treated with an intraperitoneal injection of Tat peptide alone (SEQ ID NO:85) or with δ1-1-Tat (SEQ ID NO:86), and the results are shown in FIGS. 1A-1B.

FIG. 1A is a plot of cerebral blood flow, as a percent of baseline flow, as a function of time, with delivery of the δV1-1-Tat peptide indicated by the arrow at 22 minutes. Acute delivery of δV1-1-Tat induced a 12±4% increase in cerebral blood flow (n=15; p<0.05). FIG. 1B is a scattergram plot showing the average percent change in cerebral blood flow from baseline for all animals treated with Tat alone or with δV1-1-Tat. Tat treatment caused no significant change in flow (−5±9%; n=5; n.s.).

Systemic blood pressure of the animals was also measured and did not change significantly in either treatment group, even after two weeks of sustained peptide delivery (at 13 weeks old, following 2 weeks of delivery). The average systemic blood pressure for the animals treated with δV1-1-Tat was 247±8 mmHg and for the animals treated with Tat was 246±5 mmHg.

As noted above, hypertension is a cause of chronic vascular stress, leading to loss of autoregulation, sensitivity to vasoconstrictive agents, and a reduced ability to respond to changes in arterial pressure. Weakening and loss of microvascular structure and function in hypertension translates to worsened outcome following cerebrovascular events. Delivery of δV1-1-increased cerebral blood flow by 12% in the hypertensive animals, in the absence of any additional vascular stresses (FIGS. 1A-1B). This increase in flow was not due to changes in systemic blood flow.

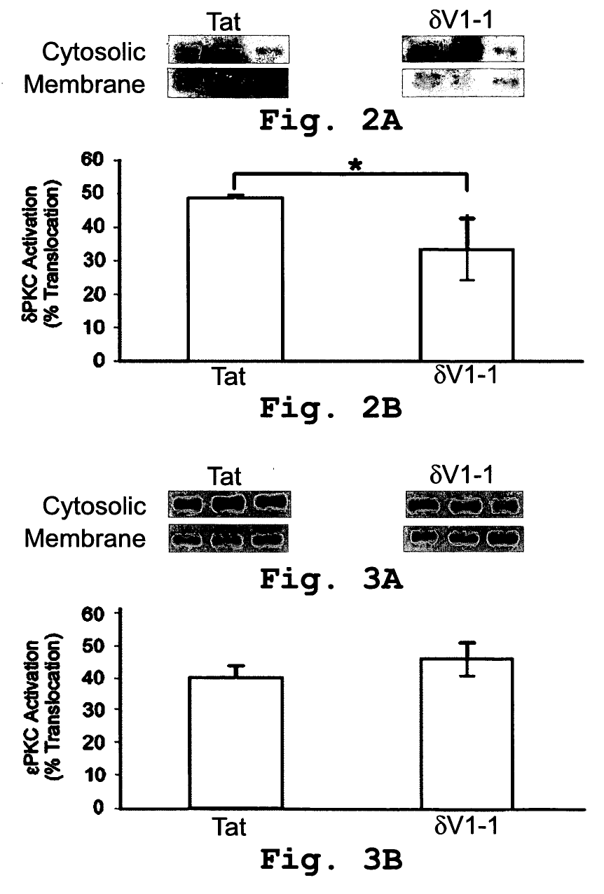

Previous reports have demonstrated that sustained delivery of δV1-1 inhibits δPKC activity in the brain of normotensive animals (Inagaki, K. et al., Circulation, 111 :44-50 (2005)). However, whether δV1-1 has similar activity in a hypertensive animal model has not been addressed. As described in Example 1, a study was conducted to determine whether chronic delivery of δV1-1 would affect δPKC activity in the brain. Dahl rats were implanted with pumps for subcutaneous delivering δV1-1-Tat or Tat control peptide for 4-6 weeks. δPKC activity in brain samples was assessed by determining the translocation of the enzyme from the soluble, cytosolic fraction, to the membrane-bound fraction, a standard marker for PKC activation (Kraft, A. S. et al., Nature, 301:621-3 (1983)). Results are shown in FIGS. 2A-2B.

FIG. 2A is a Western blot of brain tissue from animals treated with Tat alone or with δV1-1-Tat, the brain tissue fractionated into soluble (cystolic) and particulate (membrane) fractions and probed with an anti-δPKC antibody. The density of the bands was measured to assess PKC translocation, as a measure of δPKC activation. FIG. 2B is a plot of δPKC activation, expressed as a percent of translocation, for the two treatment groups. In animals that received sustained δV1-1-Tat delivery, there was a 30% reduction of δPKC translocation (activity), compared to animals chronically treated with Tat control peptide (δV1-1-Tat, 34±9% translocation; Tat control, 49±1% translocation; p<0.05; n=3).

The western blot of FIG. 2A was reprobed with an anti-εPKC antibody to assess translocation of the εPKC isozyme, and the results are shown in FIGS. 3A-3B. FIG. 3A shows the anti-εPKC antibody-probed blot for tissue from Tat treated and δV1-1-Tat treated animals. FIG. 3B shows the εPKC activation, expressed as a percent of translocation, determined from the density of the bands in FIG. 3A. Activity of εPKC was unchanged by sustained delivery of the δV1-1-Tat peptide (δV1-1-Tat treated, 40±4% translocation; Tat, 46±5% translocation; n.s.).

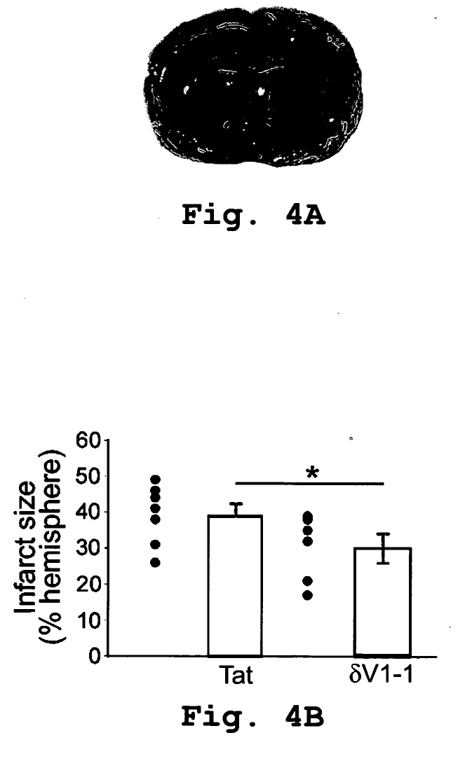

Subjects with chronic hypertension frequently use chronically administered medications (e.g. aspirin) to reduce incidence and damage from cerebrovascular events. Therefore, a sustained drug-delivery protocol was mimicked using a subcutaneously implanted osmotic pump to deliver δV1-1-Tat to evaluate whether chronic administration of the peptide was able to confer protection to hypertensive subjects against transient ischemic damage. As described in Example 1D-1E, Dahl hypertensive rats were implanted with subcutaneous pumps delivering δV1-1-Tat (SEQ ID NO:86) or Tat peptide (SEQ ID NO:85) as a control. Following 5-6 days of peptide delivery, transient focal ischemia was induced using 90 minute middle cerebral arterial occlusion, followed by 24 hours of reperfusion. Infarct size was assessed by tetrazolium chloride staining of brain tissue. The results are shown in FIGS. 4A-4B.

FIG. 4A is a representative photomicrograph of stained brain slice with cerebral infarction. FIG. 4B is a graph showing the average infarct size, measured from photomicrographs of brain slices of each animal, in animals treated with Tat alone or with δV1-1-Tat. In animals without hemorrhage, V1-1-treatment reduced infarct size by 25%, as compared with Tat controls (δV1-1, 30±4%; Tat, 39±3%; n=6-7 per group, p<0.05). Thus, chronic pump-delivery of δV1-1 inhibits δPKC activity in the brain, indicating the efficacy of this method for delivering PKC-regulating peptides in hypertensive Dahl rats. Sustained δV1-1-treatment reduced infarct size in hypertensive rats following an induced transient ischemia by 25% as compared with Tat peptide. The improved stroke outcome seen in δV1-1-treated hypertensive rats is due, at least in part, to maintenance of microvascular structure and patency, supplying improved blood-flow to the ischemic penumbra following an ischemic event. These data also demonstrate for the first time, that chronic treatment with δV1-1 peptide treatment does not cause desensitization; the neuroprotective effect of δPKC inhibition was sustained following 4-5 days of chronic δV1-1-treatment.

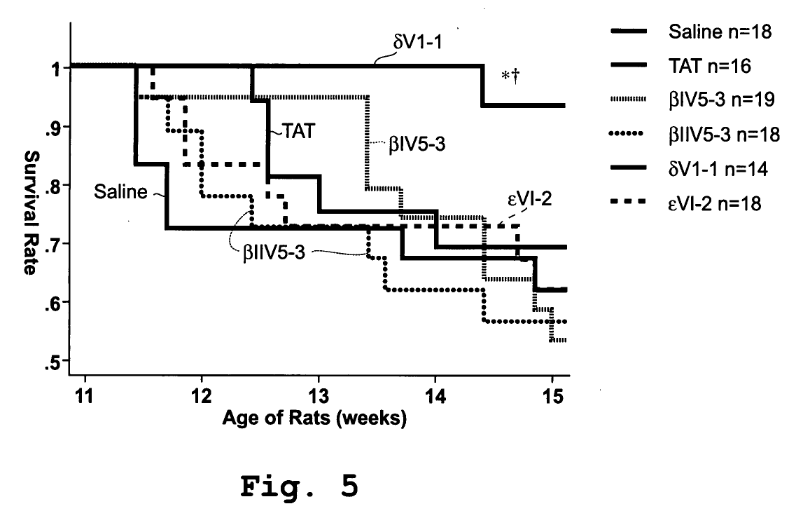

In another study, detailed in Example 2, the survival rate of hypertensive rats was monitored after induction of a stroke by occlusion of the middle cerebral artery. Prior to occlusion, the rats were treated with Tat peptide alone (SEQ ID NO:85), with δV1-1-Tat peptide (SEQ ID NO:86), with εV1-2-Tat, an eight amino acid peptide derived from εPKC (peptide εV1-2; SEQ ID NO:87; U.S. Pat. No. 6,165,977) linked to Tat (SEQ ID NO:88), or with peptides derived from the V5 domain of βPKC (SEQ ID NOS:89, 90). The results are shown in FIG. 5.

FIG. 5 shows the percentage of animals surviving as a function of animal age, in weeks, for animals treated beginning at 11 weeks of age with saline, Tat, δV1-1-Tat, or εV1-2-Tat (SEQ ID NO:89). Animals treated with a δPKC inhibitor had a marked improvement in survival rate when compared to animals treated with saline, Tat, or another isozyme-specific PKC peptide, such as εV1-2.

Treatment of hypertensive patients can be on a chronic basis, where treatment with a δPKC inhibitor is repeated daily or more than once per day on an ongoing basis. Alternatively, the treatment can be provided in the form of a sustained regimen, where the δPKC inhibitor is administered on a continuous basis, from, for example a pump, a transdermal patch, an implant, or a slow-release tablet, for a period of time.

A goal of acute stroke treatment is to save vulnerable tissue in the ischemic penumbra. This demands both protection of neurons and glia in the brain parenchyma, and preservation of microvascular structure and function, as the size and growth of the ischemic penumbra is dependent, at least in part, on the extent of compromised collateral flow. The studies discussed above show that compounds effective to inhibit δPKC can increase cerebral blood flow, even in hypertensive subjects. It will be appreciated that the treatment can be acute or chronic, depending on the patient, the severity of the conditions, and other factors apparent to a medical provider.

Preservation of microvascular structure and function correlates to survival of the ischemic penumbra following cerebrovascular events. Inhibition of δPKC improves microvascular pathology and function in chronic hypertension. This contributes to a reduction in ischemic damage following an ischemic event in a hypertensive patient. δPKC is a therapeutic target for the preservation of microcerebrovascular function following stroke, and reduces the risk of stroke and the damage due to stroke, in hypertensive patients.

As noted above, a variety of compounds can act as inhibitors of δPKC and may be utilized in the methods described herein In one embodiment, organic molecule inhibitors, including alkaloids, may be utilized. For example, benzophenanthridine alkaloids may be used, including chelerythrine, sanguirubine, chelirubine, sanguilutine, and chililutine. Such alkaloids can be purchased commercially and/or isolated from plants as known in the art and as described, for example, in U.S. Pat. No. 5,133,981.

The bisindolylmaleimide class of compounds may also be used as inhibitors of δPKC. Exemplary bisindolylmaleimides include bisindolylmaleimide I, bisindolylmaleimide II, bisindolylmaleimide III, bisindolylmaleimide IV, bisindolylmaleimide V, bisindolylmaleimide VI, bisindolylmaleimide VII, bisindolylmaleimide VIII, bisindolylmaleimide IX, bisindolylmaleimide X and other bisindolylmaleimides that are effective in inhibiting δPKC. Such compounds may be purchased commercially and/or synthesized by methods known to the skilled artisan and as described, for example, in U.S. Pat. No. 5,559,228 and Brenner, et al., Tetrahedron, 44(10):2887-2892 (1988). Anti-helminthic dyes obtained from the kamala tree and effective in inhibiting δPKC may also be utilized, including rottlerin, and may be purchased commercially or synthesized by the skilled artisan.

In certain embodiments, a protein inhibitor of δPKC may be utilized. The protein inhibitor may be in the form of a peptide. Protein, peptide, and polypeptide as used herein and as known in the art refer to a compound made up of a chain of amino acid monomers linked by peptide bonds. Unless otherwise stated, the individual sequence of the peptide is given in the order from the amino terminus to the carboxyl terminus. The protein inhibitor of δPKC may be obtained by methods known to the skilled artisan. For example, the protein inhibitor may be chemically synthesized using various solid phase synthetic technologies known to the art and as described, for example, in Williams, Paul Lloyd, et al. Chemical Approaches to the Synthesis of Peptides and Proteins, CRC Press, Boca Raton, Fla., (1997).

Alternatively, the protein inhibitor may be produced by recombinant technology methods as known in the art and as described, for example, in Sambrook et al., Molecular Cloning: A Laboratory Manual, Cold Springs Harbor laboratory, 2^{nd} ed., Cold Springs Harbor, N.Y. (1989), Martin, Robin, Protein Synthesis: Methods and Protocols, Humana Press, Totowa, N.J. (1998) and Current Protocols in Molecular Biology (Ausubel et al., eds.), John Wiley & Sons, which is regularly and periodically updated. For example, an expression vector may be used to produce the desired peptide inhibitor in an appropriate host cell and the product may then be isolated by known methods. The expression vector may include, for example, the nucleotide sequence encoding the desired peptide wherein the nucleotide sequence is operably linked to a promoter sequence.

As defined herein, a nucleotide sequence is “operably linked” to another nucleotide sequence when it is placed in a functional relationship with another nucleotide sequence. For example, if a coding sequence is operably linked to a promoter sequence, this generally means that the promoter may promote transcription of the coding sequence. Operably linked means that the DNA sequences being linked are typically contiguous and, where necessary to join two protein coding regions, contiguous and in reading frame. However, since enhancers may function when separated from the promoter by several kilobases and intronic sequences may be of variable length, some nucleotide sequences may be operably linked but not contiguous. Additionally, as defined herein, a nucleotide sequence is intended to refer to a natural or synthetic linear and sequential array of nucleotides and/or nucleosides, and derivatives thereof. The terms “encoding” and “coding” refer to the process by which a nucleotide sequence, through the mechanisms of transcription and translation, provides the information to a cell from which a series of amino acids can be assembled into a specific amino acid sequence to produce a polypeptide.

The inhibitor may be derived from an isozyme of PKC, such as δV1-1, whose amino acid sequence from Rattus norvegicus is set forth in SEQ ID NO:1 (SFNSYELGSL), representing amino acids 8-17 of rat δPKC as found in Genbank Accession No. AAH76505. Alternatively, the peptide inhibitor may be other fragments of PKC, such as δv1-2, δV1-5 and/or δV5, or some combination of δV1-1, δV1-2, δV1-5 and δV5. The amino acid sequence of δV1-2 from Rattus norvegicus is set forth in SEQ ID NO:2 (ALTTDRGKTLV), representing amino acids 35 to 45 of rat δPKC found in Genbank Accession No. AAH76505. The amino acid sequence of δV1-5 from Rattus norvegicus is set forth in SEQ ID NO:3 (KAEFWLDLQPQAKV), representing amino acids 569 to 626 of rat δPKC found in Genbank Accession No. AAH76505. The amino acid sequence of δV5 is set forth in SEQ ID NO:4 (PFRPKVKSPRPYSNFDQEFLNEKARLSYSDKNLIDSMDQS AFAGFSFVNPKFEHLLED), representing amino acids 561-626 of human δPKC found in Genbank Accession No. BM01381, with the exception that amino acid 11 (aspartic acid) is substituted with a proline.

The peptide inhibitors may include natural amino acids, such as the L-amino acids or non-natural amino acids, such as D-amino acids. The amino acids in the peptide may be linked by peptide bonds or, in modified peptides described herein, by non-peptide bonds.

A wide variety of modifications to the amide bonds which link amino acids may be made and are known in the art. Such modifications are discussed in general reviews, including in Freidinger, R. M. “Design and Synthesis of Novel Bioactive Peptides and Peptidomimetics” J. Med. Chem. 46:5553 (2003), and Ripka, A. S., Rich, D. H. “Peptidomimetic Design” Curr. Opin. Chem. Biol. 2:441 (1998). These modifications are designed to improve the properties of the peptide by increasing the potency of the peptide or by increasing the half-life of the peptide.

The potency of the peptide may be increased by restricting the conformational flexibility of the peptide. This may be achieved by, for example, including the placement of additional alkyl groups on the nitrogen or alpha-carbon of the amide bond, or by alpha modifications of the peptide, as described, for example Goodman, M. et. al. (Pure Appl. Chem., 68:1303 (1996)). The amide nitrogen and alpha carbon may be linked together to provide additional constraint (Scott et al., Org. Letts., 6:1629-1632 (2004)).

The half-life of the peptide may be increased by introducing non-degradable moieties to the peptide chain. This may be achieved by, for example, replacement of the amide bond by a urea residue (Patil et al., J. Org. Chem., 68:7274-7280 (2003)) or an aza-peptide link (Zega and Urleb, Acta Chim. Slov. 49:649-662 (2002)). Other examples of non-degradable moieties that may be introduced to the peptide chain include introduction of an additional carbon (“beta peptides”, Gellman, S. H. Acc. Chem. Res., 31:173 (1998)) or ethene unit (Hagihara et al., J. Am. Chem. Soc. 114:6568 (1992)) to the chain, or the use of hydroxyethylene moieties (Patani, G. A., Lavoie, E. J. Chem. Rev., 96:3147-3176 (1996)) and are also well known in the art. Additionally, one or more amino acids may be replaced by an isosteric moiety such as, for example, the pyrrolinones of Hirschmann et al (J. Am. Chem. Soc., 122:11037 (2000)), or tetrahydropyrans (Kulesza, A. et al., Org. Letts., 5:1163 (2003)).

Although the peptides are described primarily with reference to amino acid sequences from Rattus norvegicus, it is understood that the peptides are not limited to the specific amino acid sequences set forth in SEQ ID NOS:1-4. Skilled artisans will recognize that, through the process of mutation and/or evolution, polypeptides of different lengths and having different constituents, e.g., with amino acid insertions, substitutions, deletions, and the like, may arise that are related to, or sufficiently similar to, a sequence set forth herein by virtue of amino acid sequence homology and advantageous functionality as described herein. The terms “δV1-1 peptide”, “δV1-2 peptide”, “δV1-5 peptide” and “δV5 peptide” are used to refer generally to the peptides having the features described herein and preferred examples include peptides having the amino acid sequence of SEQ ID NO:1, SEQ ID NO:2, SEQ ID NO:3 and SEQ ID NO:4, respectively. Also included within this definition, and in the scope of the invention, are variants of the peptides which function in reducing the risk of stroke or the extent of damage subsequent to stroke, in hypertensive subjects, as described herein.

The peptide inhibitors described herein also encompass amino acid sequences similar to the amino acid sequences set forth herein that have at least about 50% identity thereto and function to reduce the risk of stroke or the extent of damage subsequent to stroke, in hypertensive subjects, or to improve survival of hypertensive subjects after stroke, as described herein. Preferably, the amino acid sequences of the peptide inhibitors encompassed in the invention have at least about 60% identity, further at least about 70% identity, preferably at least about 80% identity, more preferably at least about 90% identity, and further preferably at least about 95% identity, to the amino acid sequences, including SEQ ID NOS:1-4, set forth herein.

Percent identity may be determined, for example, by comparing sequence information using the advanced BLAST computer program, including version 2.2.9, available from the National Institutes of Health. The BLAST program is based on the alignment method of Karlin and Altschul. Proc. Natl. Acad. Sci. USA, 87:2264-2268 (1990) and as discussed in Altschul et al., J. Mol. Biol, 215:403-410 (1990); Karlin And Altschul, Proc. Natl. Acad. Sci. USA, 90:5873-5877 (1993); and Altschul et al., Nucleic Acids Res., 25:3389-3402 (1997). Briefly, the BLAST program defines identity as the number of identical aligned symbols (i.e., nucleotides or amino acids), divided by the total number of symbols in the shorter of the two sequences. The program may be used to determine percent identity over the entire length of the proteins being compared. Default parameters are provided to optimize searches with short query sequences in, for example, blastp with the program. The program also allows use of an SEG filter to mask-off segments of the query sequences as determined by the SEG program of Wootton and Federhen, Computers and Chemistry, 17:149-163 (1993).

Accordingly, fragments or derivatives of peptide inhibitors described herein may also be advantageously utilized that include amino acid sequences having the specified percent identities to SEQ ID NOS:1-4 described herein to reduce the risk of stroke or to reduce the extent of tissue damage as a result of stroke, in hypertensive patients. For example, fragments or derivatives of δV1-1, δV1-2, δV1-5 and δV5 that are effective in inhibiting δPKC and decreasing cerebral blood flow or reducing the percent of infarction, as described above in FIGS. 1-4, may also advantageously be utilized in the treatment method.

Conservative amino acid substitutions may be made in the amino acid sequences to obtain derivatives of the peptides that may advantageously be utilized in the treatment method. Conservative amino acid substitutions, as known in the art and as referred to herein, involve substituting amino acids in a protein with amino acids having similar side chains in terms of, for example, structure, size and/or chemical properties. For example, the amino acids within each of the following groups may be interchanged with other amino acids in the same group: amino acids having aliphatic side chains, including glycine, alanine, valine, leucine and isoleucine; amino acids having non-aromatic, hydroxyl-containing side chains, such as serine and threonine; amino acids having acidic side chains, such as aspartic acid and glutamic acid; amino acids having amide side chains, including glutamine and asparagine; basic amino acids, including lysine, arginine and histidine; amino acids having aromatic ring side chains, including phenylalanine, tyrosine and tryptophan; and amino acids having sulfur-containing side chains, including cysteine and methionine. Additionally, aspartic acid, glutamic acid and their amides, are also considered interchangeable herein.

Accordingly, modifications to δV1-1 that are expected to result in effective inhibition of δPKC and a concomitant reduction in the risk of stroke or the extent of damage subsequent to stroke, in hypertensive subjects, both as described herein, include the following changes to SEQ ID NO:1 shown in lower case: tFNSYELGSL (SEQ ID NO:5), aFNSYELGSL (SEQ ID NO:6), SFNSYELGtL (SEQ ID NO:7), including any combination of these three substitutions, such as tFNSYELGtL (SEQ ID NO:8). Other potential modifications include SyNSYELGSL (SEQ ID NO:9), SFNSFELGSL (SEQ ID NO:10), SNSYdLGSL (SEQ ID NO:11), SFNSYELpSL (SEQ ID NO:12).

Other possible modifications that are expected to produce a peptide that functions in the invention include changes of one or two L to I or V, such as SFNSYEiGSv (SEQ ID NO:13), SFNSYEvGSi (SEQ ID NO:14), SFNSYELGSv (SEQ ID NO:15), SFNSYELGSi (SEQ ID NO:16), SFNSYEiGSL (SEQ ID NO:17), SFNSYEvGSL (SEQ ID NO:18), aFNSYEiGSL (SEQ ID NO:19), any combination of the above-described modifications, and other conservative amino acid substitutions described herein.

Fragments and modification of fragments of δV1-1 are also contemplated, including: YELGSL (SEQ ID NO:20), YdLGSL (SEQ ID NO:21), fdLGSL (SEQ ID NO:22), YdiGSL (SEQ ID NO:23), iGSL (SEQ ID NO:24), YdvGSL (SEQ ID NO:25), YdLpsL (SEQ ID NO:26), YdLgiL (SEQ ID NO:27), YdLGSi (SEQ ID NO:28), YdLGSv (SEQ ID NO:29), LGSL (SEQ ID NO:30), iGSL (SEQ ID NO:31), vGSL (SEQ ID NO:32), LpSL (SEQ ID NO:33), LGiL (SEQ ID NO:34), LGSi (SEQ ID NO:35), LGSv (SEQ ID NO:36).

Accordingly, the term “a δV1-1 peptide” as used herein further refers to a peptide identified by SEQ ID NO:1 and to a peptide having an amino acid sequence having the specified percent identity described herein to the amino acid sequence of SEQ ID NO:1, including but not limited to the peptides set forth in SEQ ID NOS:5-19, as well as fragments of any of these peptides that retain activity for reducing the risk of stroke or the extent of damage subsequent to stroke, in hypertensive subjects, as described herein, as exemplified by but not limited to SEQ ID NOS:20-36.

Modifications to δV1-2 that are expected to result in effective inhibition of δPKC and a concomitant reduction in the risk of stroke in persons with chronic hypertension, include the following changes to SEQ ID NO:2 shown in lower case: ALsTDRGKTLV (SEQ ID NO:37), ALTsDRGKTLV (SEQ ID NO:38), ALTTDRGKsLV (SEQ ID NO:39), and any combination of these three substitutions, ALTTDRpKTLV (SEQ ID NO:40), ALTTDRGrTLV (SEQ ID NO:41), ALTTDkGKTLV (SEQ ID NO:42), ALTTDkGkTLV (SEQ ID NO:43), changes of one or two L to I, or V and changes of V to I, or L and any combination of the above. In particular, L and V can be substituted with V, L, I R and D. E can be substituted with N or Q. One skilled in the art would be aware of other conservative substitutions that may be made to achieve other derivatives of δV1-2 in light of the description herein.

Accordingly, the term “a δV1-2 peptide” as further used herein refers to a peptide identified by SEQ ID NO:2 and to a peptide having an amino acid sequence having the specified percent identity described herein to the amino acid sequence of SEQ ID NO:2, including but not limited to the peptides set forth in SEQ ID NOS:37-43, as well as fragments of any of these peptides that retain activity for increasing cerebral blood flow or reducing the area of infarct post-ischemia, in subjects with chronic hypertension, as described herein.

Modifications to δV1-5 that are expected to result in reduction of the risk of stroke or in the extent of damage subsequent to stroke, in hypertensive subjects, include the following changes to SEQ ID NO:3 shown in lower case:

rAEFWLDLQPQAKV (SEQ ID NO:44); KAdFWLDLQPQAKV (SEQ ID NO:45); KAEFWLeLQPQAKV (SEQ ID NO:46), KAEFWLDLQPQArV (SEQ ID NO;47), KAEyWLDLQPQAKV (SEQ ID NO:48), KAEFWiDLQPQAKV (SEQ ID NO:49), KAEFWvDLQPQAKV (SEQ ID NO:50), KAEFWLDiQPQAKV (SEQ ID NO:51), KAEFWLDvQPQAKV (SEQ ID NO:52), KAEFWLDLnPQAKV (SEQ ID NO:53), KAEFWLDLQPnAKV (SEQ ID NO;54), KAEFWLDLQPQAKi (SEQ ID NO;55), KAEFWLDLQPQAKI (SEQ ID NO:56), KAEFWaDLQPQAKV (SEQ ID NO:57), KAEFWLDaQPQAKV (SEQ ID NO;58), and KAEFWLDLQPQAKa (SEQ ID NO:59).

Fragments of δV1-5 are also contemplated, including: KAEFWLD (SEQ ID NO:60), DLQPQAKV (SEQ ID NO:61), EFWLDLQP (SEQ ID NO:62), LDLQPQA (SEQ ID NO:63), LQPQAKV (SEQ ID NO:64), AEFWLDL (SEQ ID NO:65), and WLDLQPQ (SEQ ID NO:66).

Modifications to fragments of δV1-5 are also contemplated and include the modifications shown for the full-length fragments as well as other conservative amino acid substitutions described herein. The term “a δV1-5 peptide” as further used herein refers to SEQ ID NO:3 and to a peptide having an amino acid sequence having the specified percent identity described herein to an amino acid sequence of SEQ ID NO:3, as well as fragments thereof that retain activity for reducing the risk of stroke in hypertensive subjects, as evidenced for example by an increase in cerebral blood flow, or to reduce the extent of tissue damage post stroke in hypertensive subjects.

Modifications to δV5 that are expected to find use in the treatment method include making one or more conservative amino acid substitutions, including substituting: R at position 3 with Q; S at position 8 with T; F at position 15 with W; V at position 6 with L and D at position 30 with E; K at position 31 with R; and E at position 53 with D, and various combinations of these modifications and other modifications that can be made by the skilled artisan in light of the description herein.

Fragments of δV5 are also contemplated, and include, for example, the following: SPRPYSNF (SEQ ID NO:67), RPYSNFDQ (SEQ ID NO:68), SNFDQEFL (SEQ ID NO:69), DQEFLNEK (SEQ ID NO:70), FLNEKARL (SEQ ID NO:71), LIDSMDQS (SEQ ID NO:72), SMDQSAFA (SEQ ID NO:73), DQSAFAGF (SEQ ID NO:74), FVNPKFEH (SEQ ID NO:75), KFEHLLED (SEQ ID NO:76), NEKARLSY (SEQ ID NO:77), RLSYSDKN (SEQ ID NO:78), SYSDKNLI (SEQ ID NO:79), DKNLIDSM (SEQ ID NO:80), PFRPKVKS (SEQ ID NO: 81), RPKVKSPR (SEQ ID NO:82), and VKSPRPYS (SEQ ID NO:83).

Modifications to fragments of δV5 are also contemplated and include the modifications shown for the full-length fragments as well as other conservative amino acid substitutions described herein. The term “a δV5 peptide” as further used herein refers to SEQ ID NO:4 and to a peptide having an amino acid sequence having the specified percent identity described herein to an amino acid sequence of SEQ ID NO:4, as well as fragments thereof that retain activity for reducing the risk of stroke, as measured for example by an increased cerebral blood flow, or in decreasing the extent of cerebral tissue damage following stroke. The inhibitors used for treatment herein may include a combination of the peptides described herein.

Other suitable molecules or compounds, including small molecules, that may act as inhibitors of δPKC may be determined by methods known to the art. For example, such molecules may be identified by their ability to translocate δPKC to its subcellular location. Such assays may utilize, for example, fluorescently-labeled enzyme and fluorescent microscopy to determine whether a particular compound or agent may aid in the cellular translocation of δPKC. Such assays are described, for example, in Schechtman, D. et al., J. Biol. Chem. 279(16):15831-15840 (2004) and include use of selected antibodies. Other assays to measure cellular translocation include Western blot analysis as described in Dom, G. W., II et al., Proc. Natl. Acad. Sci. U.S.A. 96(22):12798-12803 (1999) and Johnson, J. A. and Mochly-Rosen, D., Circ Res. 76(4):654-63 (1995).

The inhibitors may be modified by being part of a fusion protein. The fusion protein may include a protein or peptide that functions to increase the cellular uptake of the peptide inhibitors, has another desired biological effect, such as a therapeutic effect, or may have both of these functions. For example, it may be desirable to conjugate, or otherwise attach, the δV1-1 peptide, or other peptides described herein, to a cytokine or other protein that elicits a desired biological response. The fusion protein may be produced by methods known to the skilled artisan. The inhibitor peptide may be bound, or otherwise conjugated, to another peptide in a variety of ways known to the art. For example, the inhibitor peptide may be bound to a carrier peptide, such as a cell permeable carrier peptide or other peptide described herein via cross-linking wherein both peptides of the fusion protein retain their activity. As a further example, the peptides may be linked or otherwise conjugated to each other by an amide bond from the C-terminal of one peptide to the N-terminal of the other peptide. The linkage between the inhibitor peptide and the other member of the fusion protein may be non-cleavable, with a peptide bond, or cleavable with, for example, an ester or other cleavable bond known to the art.

Furthermore, in other forms of the invention, the cell permeable carrier protein or peptide that may increase cellular uptake of the peptide inhibitor may be, for example, a Drosophila Antennapedia homeodomain-derived sequence which is set forth in SEQ ID NO:84 (CRQIKIWFQNRRMKWKK), and may be attached to the inhibitor by cross-linking via an N-terminal Cys-Cys bond as discussed in Theodore, L. et al., J. Neurosci., 15:7158-7167 (1995); Johnson, J. A. et al. Circ. Res., 79:1086 (1996). Alternatively, the inhibitor may be modified by a Transactivating Regulatory Protein (Tat)-derived transport polypeptide (such as from amino acids 47-57 of Tat shown in SEQ ID NO:85; YGRKKRRQRRR) from the Human Immunodeficiency Virus, Type 1, as described in Vives, et al., J. Biol. Chem, 272:16010-16017 (1997), U.S. Pat. No. 5,804,604 and Genbank Accession No. AAT48070; or with polyarginine as described in Mitchell, et al. J. Peptide Res., 56:318-325 (2000) and Rothbard et al., Nature Med., 6:1253-1257 (2000). The inhibitors may be modified by other methods known to the skilled artisan in order to increase the cellular uptake of the inhibitors.

The inhibitors may be advantageously administered in various forms. For example, the inhibitors may be administered in tablet form for sublingual administration, in a solution or emulsion. The inhibitors may also be mixed with a pharmaceutically-acceptable carrier or vehicle. The vehicle may be a liquid, suitable, for example, for parenteral administration, including water, saline or other aqueous solution, or may be an oil or aerosol. The carrier may be selected for intravenous or intraarterial administration, and may include a sterile aqueous or non-aqueous solution that may include preservatives, bacteriostats, buffers and antioxidants known to the art. In the aerosol form, the inhibitor may be used as a powder, with properties including particle size, morphology and surface energy known to the art for optimal dispersability. In tablet form, a solid carrier may include, for example, lactose, starch, carboxymethyl cellulose, dextrin, calcium phosphate, calcium carbonate, synthetic or natural calcium allocate, magnesium oxide, dry aluminum hydroxide, magnesium stearate, sodium bicarbonate, dry yeast or a combination thereof. The tablet preferably includes one or more agents which aid in oral dissolution. The inhibitors may also be administered in forms in which other similar drugs known in the art are administered.

The inhibitors may be administered to a patient by a variety of routes. For example, the inhibitors may be administered parenterally, including intraperitoneally, intravenously, intraarterially, subcutaneously, or intramuscularly. The inhibitors may also be administered via a mucosal surface, including rectally, and intravaginally; intranasally, including by inhalation; sublingually; intraocularly and transdermally. Combinations of these routes of administration are also envisioned. A preferred mode of administration is by infusion or reperfusion.

In certain embodiments, the inhibitor described herein may be co-administered in a composition with a second therapeutic agent such as an anti-hypertensive agent or other agent suitable for treating the condition of the subject, such as a vasodilator. The second therapeutic agent and inhibitor may be administered separately or concurrently. A wide variety of therapeutic agents are envisioned for treatment.

The amount of inhibitor in the compositions will range from about 1 weight percent to about 99 weight percent, and preferably about 20 weight percent to about 70 weight percent. The amount of vasodilator in the compositions will also range from about 1 weight percent to about 99 weight percent, and preferably about 20 weight percent to about 70 weight percent. Weight percent as defined herein is the amount of the agent in mg divided by 100 grams of the composition.

A therapeutically effective amount of the inhibitor is provided. As used herein, a therapeutically effective amount of the inhibitor is the quantity of the inhibitor required to increase cerebral blood flow and/or to reduce the cell, tissue or organ damage or death that occurs due to stroke and/or reperfusion following recanalization after an ischemic stroke. This amount will vary depending on the time of administration (e.g., prior to an ischemic event, at the onset of the event or thereafter), the route of administration, the duration of treatment, the specific inhibitor used and the health of the patient as known in the art. The skilled artisan will be able to determine the optimum dosage. Generally, the amount of inhibitor typically utilized may be, for example, about 0.001 mg/kg body weight to about 3 mg/kg body weight, but is preferably about 0.01 mg/kg to about 0.5 mg/kg.

A therapeutically effective amount of the second therapeutic agent is provided either alone or co-administered as a composition with the inhibitors described herein. This therapeutically effective amount will vary as described above, especially in regard to the nature of the agent.

### III. EXAMPLES

The following examples are illustrative in nature and are in no way intended to be limiting.

### Example 1

### In vivo Delivery of δPKC Peptide to Hypertensive Rats

A. Peptide Preparation

Tat₍₄₇₋₅₇₎ carrier peptide (Tat; SEQ ID NO:85; Wender, P. A. et al., Proc Natl Acad Sci USA., 97:13003-8 (2000)) and δV1-1 (δPKC inhibitor peptide; SEQ ID NO:1) were synthesized and conjugated via a Cys S—S bond (Chen, L. et al., Proc Natl Acad Sci USA., 98:11114-9 (2001)) The δV1-1-Tat₍₄₇₋₅₇₎ conjugated peptide (SEQ ID NO:86) is referred to herein as δV1-1-Tat.

B. Laser Doppler Flowmetry

Six week old Dahl salt-sensitive rats were placed on an 8% high-salt diet to induce hypertension. Cerebral brain flow (CBF) was measured at 11 weeks, 4 weeks following high-salt diet onset. A burr hole was drilled 1 mm posterior and 6 mm lateral to bregma, corresponding to the ischemic territory. A laser Doppler transducer probe (Laserflo) was affixed above the cortex using a stereotaxic frame (Harvard Apparatus, MA), and a baseline CBF was established over a period of 20-30 minutes (1 minute intervals), Tat control peptide (n=5) or δV1-1-Tat peptide (n=15) was injected by intraperitoneal injection (0.2 mg/kg in 1 mL) and cerebral brain flow was monitored for an additional 20-30 minutes. Cerebral brain flow was also measured in the sham control animals (n=4).

Analysis of changes in cerebral brain flow was performed by comparing the baseline flow-rate at 20 minutes, to the flow-rate at 25 minutes post-treatment in all animals. Results were expressed as both a scattergram of percent flow changes in individual animals, and the mean±SEM flow in each group. Statistical analysis between Tat and δV1-1-Tat treatment groups was performed using an unpaired t-test. Results are shown in FIGS. 1A-1B.

A tail-cuff blood pressure monitor was used to measure blood pressure in Dahl rats chronically treated with δV1-1-Tat or Tat peptide using a subcutaneous osmotic pump weekly for a period of 2 weeks.

C. Western Blot Analysis

Brains from Dahl hypertensive rats that received δV1-1-Tat or Tat peptide delivery for 4 weeks, starting at 11 weeks of age, were quickly isolated and frozen. Tissue fractionation was performed to collect soluble (cytosolic) and particulate (membrane) fractions, as previously described (Johnson, J. A. et al., Life Sci., 57:1027-38(1995)). 5 μg of protein from each fraction was used forwestern blot analysis using a rabbit anti-phospho-serine 643 δPKC antibody (Santa Cruz Biotechnology; 1:500) followed with an anti-rabbit secondary antibody (Amersham). Density of δPKC bands was measured to assess translocation of PKC from the cytosol to membrane fractions, a measure of activation (Kraft, A. S. et al., Nature, 301:621-3 (1983)). Blots were then reprobed with a rabbit anti-δPKC antibody (Santa Cruz Biotechnology; 1:500), to assess translocation of the δPKC isozyme. Statistical analysis was performed using students t-test. Results are shown in FIGS. 2A-2B and 3A-3B.

D. Assessment of Brain Infarct Size in Hypertensive Rats

Animals were euthanized following 24 hours of reperfusion by isoflurane overdose. Brains were quickly removed and sliced into 3 mm coronal sections, resulting in five slices of the brain. Slices were stained using 3% triphenyl tetrazolium chloride (TTC) and both faces of each slice were photographed for infarct assessment. Relative stroke area (ratio of the infarct size relative to the ipsilateral hemisphere, corrected for edema based on measurement of the contralateral hemisphere) was measured to assess infarct size in the central three slices (two faces each; six faces total) of the five slices made from each brain (Bright, R. et al. J. Neurosci., 24:6880-88 (2004)). Results were expressed both as scaftergram of individual animals and as the mean±SEM infarct size of these six faces. Infarct size statistics between Tat and δV1-1-Tat treated groups were performed using a student's t-test. Results are shown in FIG. 4A.

E. Middle Cerebral Artery Occlusion Model

Transient focal ischemia was induced in male hypertensive Dahl salt-sensitive rats (Brookhaven Labs, NY; 290-320g) using an occluding intraluminal suture, as previously described (Bright, R. et al. J. Neurosci., 24:6880-88 (2004)). Briefly, an uncoated 30 mm long segment of 3-0 nylon monofilament suture with the tip rounded by a flame was inserted into the stump of the external carotid artery and advanced into the internal carotid artery approximately 19-20 mm from the bifurcation to occlude the ostium of the middle cerebral artery (MCA). The ischemic period in the Dahl hypertensive rat was 90 minutes due to high mortality using a 120 minute occlusion period. Following the ischemic period the suture was removed and the animal was allowed to recover. In the hypertensive model, peptides were delivered chronically using an Alzet subcutaneous pump (Alza, Calif.; 1 mM, 5 μL/hr), implanted dorsally, for a period spanning 4-5 days prior to surgery until sacrifice at 24 hours following surgery (Inagaki, K. et al., Circulation, 111:44-50 (2005)). Results are shown in FIG. 4B.

### Example 2

### Survival Rate of Hypertensive Rats Following Stroke

Dahl salt-sensitive rats were fed with 8% salt diet from 6 weeks old. Rats had high systemic blood pressure at 11 weeks old. Rats were treated with saline, a TAT carrier peptide (4.5 nmol/hour, SEQ ID NO:85), a βI-PKC inhibitor conjugated to Tat (βI-V5-3, 4.5 nmol/hour, peptide derived from SEQ ID NO:90), a βII-PKC inhibitor conjugated to Tat (βII-V5-3, 4.5 nmol/hour, peptide derived from SEQ ID NO:91), a δ-PKC inhibitor (δV1-1-Tat, SEQ ID NO:86, 1.5 nmol/hour) or an ε-PKC inhibitor (εV1-2-Tat, SEQ ID NO:89, 4.5 nmol/hour) subcutaneously using osmotic pumps for 4 weeks from 11 weeks old. The survival rate of the animals was monitored from hypertension-induced stroke and systemic blood pressure. The results are shown in FIG. 5.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

It is claimed:

1. A method for reducing the risk of stroke in a subject with hypertension, comprising: administering an inhibitor of delta protein kinase C (PKC) to the subject.

2. The method of claim 1, wherein said subject has chronic hypertension.

3. The method of claim 1, wherein said subject has acute hypertension.

4. The method of claim 1, wherein said inhibitor is a peptide.

5. The method of claim 4, wherein said peptide has an amino acid sequence having at least about 50% identity to the amino acid sequence of delta V1-1 set forth in SEQ ID NO:1.

6. The method of claim 4, wherein said peptide is delta V1-1 having an amino acid sequence as set forth in SEQ ID NO:1.

7. The method of claim 4, wherein said peptide is linked to a moiety effective to facilitate transport across a cell membrane.

8. The method of claim 7, wherein said peptide is modified to include at least one terminal cysteine residue.

9. The method of claim 4, wherein said peptide is Cys-Cys bonded to a carrier peptide selected from poly-Arg, Tat, or the Drosophila Antennapedia homeodomain.

10. The method of claim 4, wherein said administering includes administering a pharmaceutical formulation comprising a pharmaceutically-acceptable excipient and a peptide inhibitor of delta PKC.

11. The method of claim 10, wherein said administering is parenteral administration.

12. The method of claim 11, wherein said parenteral administration is subcutaneous.

13. The method of claim 12, wherein said subcutaneous administration is via an implanted osmotic pump.

14. The method of claim 10, wherein said administering is on a chronic or sustained basis.

15. A method for improving survival from stroke in a subject with chronic hypertension, comprising: administering an inhibitor of delta protein kinase C (PKC) to the subject.

16. The method of claim 15, wherein said inhibitor is a peptide.

17. The method of claim 16, wherein said peptide has an amino acid sequence having at least about 50% identity to the amino acid sequence of delta V1-1 set forth in SEQ ID NO:1.

18. The method of claim 16, wherein said peptide is delta V1-1 having an amino acid sequence as set forth in SEQ ID NO:1.

19. The method of claim 16, wherein said peptide is linked to a moiety effective to facilitate transport across a cell membrane.

20. The method of claim 19, wherein said peptide is modified to include at least one terminal cysteine residue.

21. The method of claim 16, wherein said administering includes administering a pharmaceutical formulation comprising a pharmaceutically acceptable excipient and a peptide inhibitor of delta PKC.

22. The method of claim 21, wherein said administering is parenteral administration.

23. The method of claim 22, wherein said parenteral administration is subcutaneous.

24. The method of claim 23, wherein said subcutaneous administration is via an implanted osmotic pump.
